(19) United States
(12) Patent Application Publication
(10) Pub. No.: US 2008/0095752 A1
(43) Pub. Date: Apr. 24, 2008

(54) Method for extending the shelf-life of powdered nutritional formulations which contain viable probiotics

(76) Inventors: Win-Chin Chiang, Newburgh, IN (US); Bryon W. Petschow, Ewing, NJ (US); Adrie Baars, Hengelo (NL); Yung H. Lee, Evansville, IN (US); Luis Jose Montelongo, Lawrence, KS (US); Nagendra Rangavajla, Newburgh, IN (US)

Correspondence Address: BRISTOL-MYERS SQUIBB COMPANY - MEAD JOHNSON, 2400 WEST LLOYD EXPRESSWAY, PATENT DEPARTMENT, EVANSVILLE, IN, 47721, US

(21) Appl. No.: 11/584,302

(22) Filed: Oct. 20, 2006

(51) Int. Cl. A61K 35/74; A61K 31/715

(52) U.S. Cl. 424/93.45; 514/61

(57) ABSTRACT

The invention involves a method for extending the shelf life of a powdered nutritional formulation that contains LGG by reducing and maintaining a threshold water activity or moisture content in the powdered LGG-containing formulation.

### BACKGROUND OF THE INVENTION

(1) Field of the Invention

The present invention relates to a method for extending the shelf-life of powdered nutritional formulations which contain viable probiotics.

(2) Description of the Related Art

There are at least 400 different species of bacteria that inhabit the human digestive system, often referred to as the gut flora. The gut flora are necessary to break down food remains that have not yet been digested as well as to discourage harmful bacteria and yeasts from invading the body. Some of these species are beneficial and others are potentially harmful. A balance between the two is vital for health and well-being.

Illness, poor diet, stress, aging, infection by food poisoning and the use of medications can each disturb the balance between the beneficial and harmful bacteria. An overabundance of harmful bacteria can cause diarrhea, infections, liver damage, carcinogenesis and intestinal putrefaction. In contrast, beneficial bacteria can inhibit the growth of harmful bacteria, stimulate the immune functions, reduce gas distention problems, improve digestion, absorb essential nutrients, and synthesize vitamins.

Probiotics are microbial cell preparations or components of microbial cells that have a beneficial effect on the health and well being of the host. These beneficial bacteria have various health benefits for consumers, including inhibition of bacterial pathogens, reduction of colon cancer risk, stimulation of immune response, and reduction of serum cholesterol levels.

While most live bacteria that are ingested die when they reach the acidic conditions of the stomach, probiotic bacteria are generally resistant to gastric, bile and pancreatic fluids and are able to reach the colon alive. Probiotics attach to the wall of the intestine where they increase the number of beneficial bacteria and fight against harmful bacteria, maintaining a balance between the two. Probiotics also produce short chain fatty acids which reduce the pH in the gut. A reduced pH in the gut contributes to protection of the gut mucosal cells, suppression of undesirable microbes in the gut, suppression of gut infections, increased uptake of calcium and magnesium, and stimulated immune functions.

While there are several ways to administer probiotics to consumers, one convenient way is to add probiotics to compositions that would normally be consumed. For example, probiotics are sometimes administered through a powdered nutritional formulation, such as a powdered protein supplement, a powdered milk, a powdered baby food, or a powdered infant formula. In order to obtain the desired health benefits, however, the probiotic must be selected carefully and added to the powdered formulations in sufficient amounts to ensure that the recommended dose is consumed.

Whether administered through a protein powder, powdered milk, powdered baby food, or powdered infant formula, the formulations must be processed and handled in a manner that maintains the viability of the probiotic microorganisms during the manufacturing process and during the time such formulations spend on the shelf waiting for sale and consumption. Unfortunately, many probiotics that are added to powdered nutritional formulations are killed during shipping, distribution, or the manufacturing process, or simply die while the product sits on the shelf for extended periods.

Because nutritional formulations are often commercially available in large quantities, a relatively long shelf-life is required for the product. The probiotics must maintain viability at least until the product is consumed in the normal course of administration. One factor that reduces the shelf-life of probiotic formulations is temperature. Probiotics are living organisms that die at a much faster rate when not refrigerated. In order to prevent the death of the microorganisms in these products, many probiotic-containing powdered nutritional products recommend constant refrigeration or freezing.

Another factor that reduces the shelf-life of probiotic formulations is water activity or moisture content. Water activity is the ratio of the vapor pressure of water in a material to the vapor pressure of pure water at the same temperature. It describes the continuum of energy states of the water in a system. Moisture content can be defined as percentage weight of water in relation to the dry weight of the product.

Exposure to even a minimum amount of moisture can rapidly destroy the potency of probiotics. An especially difficult technical barrier to extending the shelf-life of nutritional formulations that contain probiotics is the relatively high moisture content of the ingredients that make up the nutritional product.

A suitable method for extending the shelf-life of a powdered probiotic-containing nutritional formulation without encapsulating, lyophilizing or using matrices remains very limited. Accordingly, it would be useful to provide a method for extending the shelf-life of powdered nutritional formulations that contain viable probiotics.

### SUMMARY OF THE INVENTION

Briefly, therefore, the present invention is directed to a novel method for extending the shelf-life of a powdered nutritional formulation that contains LGG to at least 15 months, the method comprising reducing the water activity of the LGG-containing formulation to less than about 0.16 and maintaining the temperature of the formulation at or below 25° C.

The present invention is also directed to a novel method for extending the shelf-life of a powdered nutritional formulation that contains LGG to at least 15 months, the method comprising reducing the moisture content of the LGG-containing formulation to less than about 2.3% and maintaining the temperature of the formulation at or below 25° C.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

The term extended “shelf-life” as used herein means a period that a product can be stored without the quality falling below a certain minimum acceptable level. The minimum acceptable level for the probiotic-containing powdered nutritional formulation of the present invention requires that the composition maintain substantially the same physical and chemical properties, e.g. taste, smell, color, and the like, for at least 15 months and that the compositions contain viable probiotics in an amount of at least 80% of the inoculated amount when the compositions are stored at or below 25° C.

The term “aseptic conditions” as used herein means an atmosphere essentially free of microorganisms and includes the filling of a commercially sterilized powdered nutritional formulation into pre-sterilized containers followed by aseptic hermetical sealing with a pre-sterilized closure in an atmosphere essentially free of microorganisms.

“Infant formula” as used herein means a composition that satisfies the nutrient requirements of an infant by being a substitute for human milk.

As used herein, the term “probiotic” means a live microbial feed supplement which beneficially affects the host animal by improving its intestinal microbial balance.

The term “prebiotic” means any non-digestible food ingredient that beneficially affects the host by selectively stimulating the growth and/or activity of bacteria, including probiotic bacteria, in the colon, with the effect of improving the host's health.

In accordance with the present invention, a novel method has been discovered for extending the shelf-life of a powdered nutritional formulation that contains LGG to at least 15 months. The method comprises reducing the water activity of the LGG-containing formulation to less than about 0.16 and maintaining the temperature of the formulation at or below 25° C. In a particular embodiment, the method comprises reducing the water activity of the LGG-containing formulation to less than about 0.14 and maintaining the temperature of the formulation at or below 25° C.

The method also comprises reducing the moisture content of the LGG-containing formulation to less than about 2.3% and maintaining the temperature of the formula at or below 25° C. In a particular embodiment, the method comprises reducing the moisture content of the LGG-containing formulation to less than about 2.1% and maintaining the temperature of the formula at or below 25° C.

In an embodiment, this method provides a shelf-life in the LGG-containing formulation of at least 15 months. In another embodiment, this method may provide a shelf-life in the LGG-containing formulation of at least 18 months. In yet another embodiment, this method may provide a shelf-life in the LGG-containing formulation of at least 21 months.

With the extended shelf-life provided by the present invention, it is not necessary to add chemical preservatives to the nutritional formulation or lyophilize, encapsulate, or provide a matrix for the LGG in order to preserve its viability. Also, due to the extended shelf-life provided by the present invention, the LGG-containing nutritional formulation does not require constant refrigeration or freezing. The formulation is thus suitable for shipping and distribution. The formulation can be purchased in larger, more convenient or cost-effective quantities, as the viability of the organisms will be maintained for longer periods of time.

LGG is a probiotic strain isolated from healthy human intestinal flora. It was disclosed in U.S. Pat. No. 5,032,399 to Gorbach, et al., which is herein incorporated in its entirety, by reference thereto. LGG is resistant to many antibiotics, stable in the presence of acid and bile, and attaches avidly to mucosal cells of the human intestinal tract. It survives for 1-3 days in most individuals and up to 7 days in 30% of subjects. In addition to its colonization ability, LGG also beneficially affects mucosal immune responses. LGG is deposited with the depository authority American Type Culture Collection under accession number ATCC 53103.

In an embodiment of the present invention, additional probiotics may be added to the powdered nutritional formulation. Any probiotic known in the art will be acceptable in this embodiment. In a particular embodiment, the probiotic is chosen from the group consisting of Lactobacillus, Bifidobacterium and combinations thereof.

To prepare the present invention, LGG microorganisms can be cultivated using processes conventional in the art. The LGG can be used in its cultivated state or it may be processed as desired by purifying, concentrating or finishing it to produce various preparations.

The amount of LGG in the powdered nutritional formulation is an amount sufficient to provide or deliver the desired probiotic effect. A sufficient amount of LGG may vary within a broad range, depending on, for example, the total amount of cells of the LGG, the total daily dose desired, and on other properties and ingredients of the product. A daily dose of the powdered nutritional formulation of the present invention can comprise about 10⁶ to 10¹² colony forming units (cfu) of LGG per gram formulation. In another embodiment, a daily dose of the powdered nutritional formulation of the present invention can comprise about 10⁷ to 10¹⁰ cfu of LGG per gram formulation. In yet another embodiment, a daily dose of the powdered nutritional formulation of the present invention can comprise about 10⁸ to 10¹¹ cfu of LGG per gram formulation. In a particular embodiment, a daily dose of the powdered nutritional formulation of the present invention can comprise about 10⁹ cfu of LGG per gram formulation.

In one embodiment, the product maintains at least 10⁶ cfu/g LGG per gram formulation for a period of at least about 15 months. In another embodiment, the product maintains at least 10⁶ cfu/g LGG per gram formulation for a period of at least about 18 months. In yet another embodiment, the product maintains at least 10⁶ cfu/g LGG per gram formulation for a period of at least about 21 months.

In a particular embodiment, the present invention comprises the addition of at least one prebiotic to the composition. In this embodiment, any prebiotic known in the art may be added. In a particular embodiment the prebiotic can be selected from the group consisting of insulin, fructo-oligosaccharide, gluco-oligosaccharide, galacto-oligosaccharide, isomalto-oligosaccharide, xylo-oligosaccharide, polydextrose and lactulose.

Optionally, the LGG and prebiotic utilized in the present invention can be commercially purchased as a premixed powder. Commercial sources for products that contain both LGG and various prebiotics are known in the art. In another embodiment, the LGG and the prebiotic can be purchased separately and intermixed using any suitable method in the art. In this embodiment, it is preferred that the particle sizes of the LGG and prebiotic are the same or similar.

The powdered nutritional formulation of the present invention can be purchased commercially or can be individually prepared. If individually prepared, the nutritional formula may be prepared in any suitable manner known in the art. For example, U.S. Pat. No. 6,506,422 to Masson, et al., incorporated herein by reference, discloses a method for preparation of a nutritional formula. A similar method can be utilized to prepare a powdered nutritional formulation for the present invention.

In an embodiment, the infant formula for use in the present invention is nutritionally complete and contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, partially hydrolyzed protein, amino acids, and the like. In one embodiment, the protein is a combination of whey protein and casein in a ratio of 60:40. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, soybean oil, palmolein, coconut oil, medium chain triglyceride oil, high oleic sunflower oil, high oleic safflower oil, and the like.

Conveniently, commercially available infant formula can be used. For example, Enfalac, Enfamil®, Enfamil® Premature Formula, Enfamil®) with Iron, Lactofree®, Nutramigen®, Pregestimil®), and ProSobee® (available from Mead Johnson & Company, Evansville, Ind., U.S.A.) may be supplemented with suitable levels of LGG and used in practice of the method of the invention.

According to a particular embodiment, the powdered nutritional formulation of the present invention is a powdered milk, a powdered nutritional supplement, a powdered infant formula, or a powdered baby food. In a specific embodiment, the powdered nutritional formulation is an infant formula.

Any method known in the art for reducing the water activity or moisture content of the composition can be used in the present invention. In one embodiment of the present invention, the method for reducing the water activity or moisture content of the composition comprises incorporating an amount of desiccant into the packaging structure that contains the powdered nutritional formulation and LGG in order to control the moisture content of the powdered product. Any type of desiccant is suitable for use in this embodiment. In a particular embodiment, dehydrated metal aluminosilicate is used as the desiccant material. Such a desiccant, Tri-Sorb®, is commercially available from Texas Technologies, located in Leander, Tex.

In yet another embodiment of the present invention, the method for reducing the water activity or moisture content of the composition comprises introducing a purging agent into the blending or mixing process. In this embodiment, the powdered nutritional formulation and LGG are introduced into a mixer or blender. In one embodiment, the blender is a conical screw blender. A stream of a purging agent can then be introduced at or near the base of the conical screw blender. In some embodiments, multiple streams of purging agent can be introduced at or near the base of the blender. For example, in one embodiment, two gas injection lines are utilized. In another embodiment, three injection lines are utilized.

In some embodiments, the multiple injection lines are located at various heights within the vessel. In this embodiment, one injection line could be located at the base of the vessel, a second injection line located at a position which is about ⅙ of the height of the vessel, measured from the base of the vessel, and a third injection line located at a position which is about ⅓ of the height of the vessel, measured from the base of the vessel.

In particular embodiments, the multiple injection lines are located at varying positions about the circumference of the blender. In another embodiment, the injection lines are located equidistant from one another. By introducing several streams of a purging agent into a conical blender's vessel, the device becomes extremely effective in mixing, drying, reducing shear stress, reducing friction and removing oxygen from the atmosphere within the vessel.

In a particular embodiment, the purging agent is selected from the group nitrogen and carbon dioxide, but can be any agent that removes or replaces oxygen. The purging agent may be introduced in an amount which is sufficient to move the blending material upward and form a local spouting bed motion.

In yet another embodiment of the present invention, the powdered nutritional formulation and the LGG are each individually dried and are then dry-blended together to control the water activity or moisture content of the powdered product. Conventional drying processes for powdered nutritional formulations include dry-mixing, spray drying, agglomeration, or any combination of those drying processes.

In another embodiment of the present invention, the methods recited above are used in combination with each other. For example, a powdered nutritional formulation and LGG are dried separately and dry-blended together and then placed into a sealed package that contains a desiccant material. In another example, a purging agent is utilized in the mixing process of the powdered nutritional formulation and LGG and then the mixture is placed into a package that contains a desiccant material. In yet another example, a powdered nutritional formulation and powdered LGG are dried separately and then dry-blended together in the presence of a purging agent. In another example, a powdered nutritional formulation and LGG are dried separately, dry-blended together with a stream of a purging agent, and then placed into sealed package that contains a desiccant material.

In an embodiment, the composition is placed in sterile containers and sealed with sterile closures under aseptic conditions. The containers can be flushed under aseptic conditions with a sterile, inert gas to remove oxygen from the container just before sealing. The sterile, inert gas can be nitrogen or carbon dioxide. The removal of oxygen prevents the death of many facultatively anaerobic microorganisms. If air remains in the container during storage, oxygen toxicity can result in a significant loss in concentration of the probiotics during production and storage.

Any container and closure capable of maintaining a sealed, aseptic environment during processing and storage can be used to store the powdered nutritional formulation. Acceptable examples include, but are not limited to, glass bottles, composite metal cans, paper cartons, and plastic bottles. Preferably, the containers have low oxygen permeability, are resistant to light transmission, and maintain their integrity during handling.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

This example illustrates the determination of the death rate constant, k, for LGG. The goal was to determine the optimal water activity and moisture content of a LGG-containing powdered infant formula in order for it to maintain its shelf-life for at least 18 months. In order to do so, the inventors first determined the death rate constant (k) for LGG.

The destruction of microorganisms usually follows first order kinetics, which can be expressed as follows:

$$\frac{d N}{d t} = - kN \qquad ( 1 )$$

where
N: number of survivors
t: time, in weeks
k: death rate constant.

### Integrating equation (1) between time=0 and time=t, gives the following

N=No exp(−kt) (2)

where Nₒ is the initial cell count.
Equation (2) can be expressed as follows:

$$ln ( \frac{N}{{N}_{o}} ) = - kt \qquad ( 3 )$$

By plotting ln(N/Nₒ) versus storage time, t, the slope of the straight line can be obtained, which is the death rate constant, k, for LGG. Using equation 3, this calculation is shown below.

$$ln ( \frac{1 \times {10}^{6}}{5 \times {10}^{7}} ) = - 3.912 = - k ( 78 )$$

According to the equation, k=0.05015/week. Thus, in order to allow one and a half log cycle of LGG count reduction, e.g., from 5.0×10⁷ cfu/g to 1.0×10⁶ in 18 months (78 weeks), the k value of LGG has to be less than or approximately equal to 0.05/week.

### EXAMPLE 2

This example illustrates the determination of the optimal moisture content and water activity of an LGG-containing powdered infant formula in order for it to maintain its shelf-life. In this example, three major ingredients in Nutramigen® infant formula were intermixed: Nutramigen® powder base, corn syrup solids, and protein hydrolysate. The component ingredients of Nutramigen® powder base are listed in Table 1.

TABLE 1
Component Ingredients of Nutramigen ® Powder Base

| Ingredient, unit | Per 100 kg base |
|---|---|
| Corn Syrup Solids, kg | 43.135 |
| Palm Olein Oil, kg | 16.2 |
| Modified Corn Starch, kg | 16.143 |
| Coconut Oil, kg | 7.2 |
| Soy Oil, kg | 7.2 |
| High Oleic Sunflower Oil, kg | 5.4 |
| Calcium Phosphate Dibasic, kg | 2.286 |
| Potassium Citrate, kg | 0.87 |
| Potassium Chloride, kg | 0.66 |
| Calcium Citrate, kg | 0.614 |
| Choline Chloride, kg | 0.154 |
| Magnesium Oxide Light, kg | 0.118 |
| L-Carnitine, g | 19.8 |
| Sodium Iodide, g | 0.119 |

An initial amount of LGG was added to the Nutramigen® base, corn syrup solids, and protein hydrolysate mixture in order to prepare a product containing 6.25×10⁸ cfu/g product. The moisture content and water activity of the mixture were initially measured at ambient conditions using an AquaLab Water Activity meter. The oven-drying method was used in a quality control laboratory to measure the moisture content and water activity of the composition. This involved holding the samples in an oven at 70° C. under at least 24 inches of vacuum for four hours. The initial moisture content was determined to be 2.7% and the water activity was about 0.2.

The LGG-containing powdered infant formula was then placed in sealed desiccators and different quantities of Tri-sorb® desiccant packs were inserted into the desiccators to reduce and control the water activity and moisture content of the formula. The formula was stored in this manner for six months.

Over the six-month storage period, the LGG count, moisture content, and water activity of the composition were tested in one-month increments. The average values for moisture content and water activity were calculated and recorded for each Tri-sorb®) pack. With a 5 g Tri-sorb®pack, the moisture content of the composition was reduced to 2.3±0.2% and the water activity (A_{w}) of the composition was reduced to 0.16. With a 15 g Tri-sorb®) pack, the moisture content was reduced to 2.0±0.2% and the water activity of the composition was reduced to 0.11 A_{w}. With a 25 g Tri-sorb® pack, the moisture content and water activity of the composition was reduced to 1.5%±0.2% and 0.08 A_{w}, respectively.

The enumeration of LGG count was conducted as follows: 20 g of powdered product were transferred to a sterile stomacher bag and mixed with 180 mL of sterile peptone-saline diluent for 60 seconds at 200 excursions per minute. The primary dilution was serially diluted so the final dilution was 10⁻⁸. This procedure was followed three times (three replicates) for increased accuracy. The 5th, 6th, 7th and 8th dilutions were plated and incubated at 37° C. for 72 hours. The results were reported as colony forming units (cfu) per gram of product. The final LGG count was 3.93×10⁸ cfu/g.

The initial and final LGG counts for the varying Tri-sorb®) packs were then plotted against the storage time based on equation (3). It was found that the 5 g of Tri-sorb® desiccant was able to give the LGG death rate constant of 0.019/week based on 6 months (24 weeks) of storage data. All the above data were based on 25° C.

Because 5 g of Tri-sorb®) desiccant provided an LGG death rate constant of less than 0.05/week (0.019/week), it was determined that 2.3% was the critical moisture content and 0.16 A_{w} was the critical water activity for an LGG-containing powdered infant formula.

### EXAMPLE 3

This example illustrates the determination of the shelf-life of an LGG-containing powdered infant formula having a moisture content of 2.1% and water activity of 0.14 A_{w}. The powdered infant formula used in this example was Nutramigen®, available from Mead Johnson Nutritionals, Evansville, Ind. The composition of Nutramigen® powder is listed in Table 2.

TABLE 2
Nutramigen ® Ingredients

| | Per 100 Calories |
|---|---|
| Ingredients | (5 fl oz) |
| Protein, g | 2.8 |
| Fat, g | 5.3 |
| Carbohydrate, g | 10.3 |
| Water, g | 133 |
| Vitamin A, IU | 300 |
| Vitamin D, IU | 50 |
| Vitamin E, IU | 2 |
| Vitamin K, μg | 8 |
| Thiamin (Vitamin B1), μg | 80 |
| Riboflavin (Vitamin B2), μg | 90 |
| Vitamin B6, μg | 60 |
| Vitamin B12, μg | 0.3 |
| Niacin, μg | 1000 |
| Folic acid (folacin), μg | 16 |
| Pantothenic acid, μg | 500 |
| Biotin, μg | 3 |
| Vitamin C (ascorbic acid), mg | 12 |
| Choline, mg | 12 |
| Inositol, mg | 17 |
| Carnitine, mg | 2 |
| Taurine, mg | 6 |
| Calcium, mg | 94 |
| Phosphorus, mg | 63 |
| Magnesium, mg | 11 |
| Iron, mg | 1.8 |
| Zinc, mg | 1 |
| Manganese, μg | 25 |
| Copper, μg | 75 |
| Iodine, μg | 15 |
| Selenium, μg | 2.8 |
| Sodium, mg | 47 |
| Potassium, mg | 110 |
| Chloride, mg | 86 |

The three major components of Nutramigen®) infant formula are Nutramigen® base, corn syrup solids, and protein hydrolysate. In this example, the Nutramigen® base contained 2.0% moisture, the corn syrup solids contained 1.7% moisture, and the protein hydrolysate contained 2.1% moisture. An initial amount of LGG was added to the mixture in order to prepare a product containing 5.7×10⁷ cfu/g product. The moisture content of the composition was 2.1% and the water activity of the composition was 0.14 A_{w}.

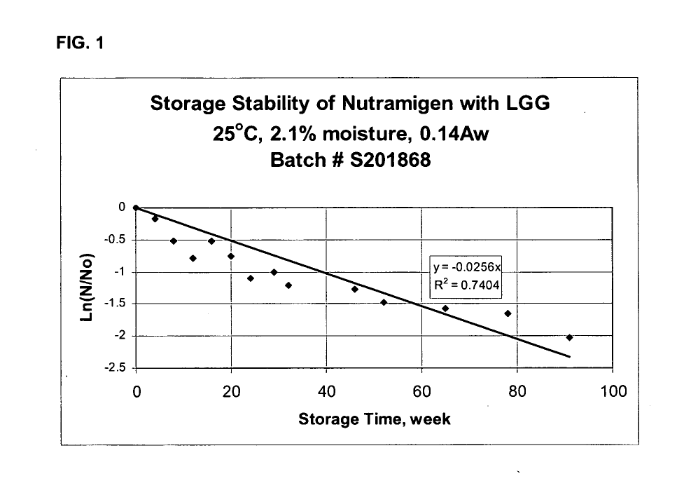

The composition was stored at 25° C. for 21 months (91 weeks). It was determined that after 21 months, the moisture content of the composition was 2.1% and the water activity of the composition was 0.14 A_{w}. The final LGG count was determined to be 7.6×10⁶ cfu/g product. The initial and final LGG counts were then plotted against the storage time based on equation (3). As shown in FIG. 1, the LGG decaying rate constant is less than 0.05/week. Specifically, the decaying rate constant is 0.0256/week.

This actual storage data shows that shelf life of the LGG-containing powdered infant formula at 25° C. with a moisture content of 2.1% and a water activity of 0.14 A_{w} can be 21 months or longer before the LGG count reduction reaches one and half log cycle.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. For example, while methods for the production of a commercially sterile liquid nutritional supplement made according to those methods have been exemplified, other uses are contemplated. Therefore, the spirit and scope of the appended claims should not be limited to the description of the preferred versions contained therein.

What is claimed is:

1. A method for extending the shelf-life of an LGG-containing powdered nutritional formulation to at least 15 months, the method comprising reducing the water activity of the LGG-containing formulation to less than about 0.16 and maintaining the temperature of the formulation at or below 25° C.

2. The method according to claim 1, wherein the water activity is reduced to less than about 0.14.

3. The method according to claim 1, wherein the shelf-life of the LGG-containing formulation is extended to at least 18 months.

4. The method according to claim 1, wherein the shelf-life of the LGG-containing formulation is extended to at least 21 months.

5. The method according to claim 1, wherein the nutritional formulation additionally comprises another probiotic organism.

6. The method according to claim 5, wherein the probiotic organism is selected from the group consisting of Lactobacillus, Bifidobacterium, and combinations thereof.

7. The method according to claim 1, wherein the LGG-containing formulation additionally comprises a prebiotic.

8. The method according to claim 7, wherein the prebiotic is selected from the group consisting of inulin, fructo-oligosaccharide, gluco-oligosaccharide, galacto-oligosaccharide, isomalto-oligosaccharide, xylo-oligosaccharide, polydextrose, and lactulose.

9. The method according to claim 1, wherein the powdered nutritional formulation comprises a powdered nutritional supplement, a powdered milk, a powdered baby food, or a powdered infant formula.

10. The method according to claim 1, wherein reducing the water activity of the LGG-containing formulation comprises one or more of the following steps:
a) providing a packaging structure to contain said LGG-containing formulation and incorporating a desiccant material into said packaging structure; or
b) blending the powdered nutritional formulation and the LGG in a blender and introducing a stream of a purging agent at the base of said blender; or
c) drying the individual components of the powdered nutritional formulation, drying the LGG, and dry-blending the components together.

11. The method according to claim 10, wherein said desiccant material is dehydrated metal aluminosilicate.

12. The method according to claim 10, wherein the purging agent is selected from the group consisting of nitrogen and carbon dioxide.

13. The method according to claim 10, further comprising placing the powdered LGG nutritional formulation into a sterile container and sealing the container with a sterile closure in an atmosphere essentially free of microorganisms.

14. The method according to claim 10, wherein the container is flushed under aseptic conditions with a sterile inert gas before being sealed.

15. A method for extending the shelf-life of an LGG-containing powdered nutritional formulation to at least 15 months, the method comprising reducing the moisture content of the LGG-containing formulation to less than about 2.3% and maintaining the temperature of the formulation at or below 25° C.

16. The method according to claim 15, wherein the moisture content is reduced to less than about 2.1%.

17. The method according to claim 15, wherein the shelf-life of the LGG-containing formulation is extended to at least 18 months.

18. The method according to claim 15, wherein the shelf-life of the LGG-containing formulation is extended to at least 21 months.

19. The method according to claim 17, wherein reducing the water activity of the LGG-containing formulation comprises one or more of the following steps:
a) providing a packaging structure to contain said LGG-containing formulation and incorporating a desiccant material into said packaging structure; or
b) blending the powdered nutritional formulation and the LGG in a blender and introducing a stream of a purging agent at the base of said blender; or
c) drying the individual components of the powdered nutritional formulation, drying the LGG, and dry-blending the components together.
